(19) United States
(12) Patent Application Publication
(10) Pub. No.: US 2024/0058207 A1
(43) Pub. Date: Feb. 22, 2024

(54) IRRADITION HEAD FOR A MASSAGE GUN

(71) Applicant: SHENZHEN KAIYAN MEDICAL EQUIPMENT CO., LTD, Shenzhen (CN)

(72) Inventors: ALAIN DIJKSTRA, Amstelveen (NL); Chen Shoufeng, Shenzhen (CN)

(73) Assignee: SHENZHEN KAIYAN MEDICAL EQUIPMENT CO., LTD, Shenzhen (CN)

(21) Appl. No.: 18/047,666

(22) Filed: Oct. 19, 2022

(30) Foreign Application Priority Data
Aug. 18, 2022 (CN) 202222168603.5

(51) Int. Cl. A61H 23/00; A61N 1/36; A61N 2/00; A61N 5/06; A61H 23/02

(52) U.S. Cl. CPC A61H 23/006; A61N 1/36014; A61N 2/002; A61N 5/0613; A61H 23/02; A61H 2201/10; A61H 2201/1669; A61H 2201/5097; A61H 2201/503; A61H 2201/5046; A61H 2230/25; A61H 2201/5071; A61H 2201/0207; A61H 2201/0214; A61H 2201/0153; A61N 2005/0663; A61N 2005/0652; A61N 2005/0644; A61H 2201/0157

(57) ABSTRACT

An irradiation head adapted to be detachably attached to a massage gun, the irradiation head being configured to reciprocate in forward and backward motion, comprises one or more irradiation sources, and a converter configured to convert the forward and backward motion of the irradiation head into electrical energy. The one or more irradiation sources are configured to be activated by the converted electrical energy, wherein the converter includes a coil based linear alternator, the coil based linear alternator including, a coil wrapped around a hollow cylindrical body, and a magnet disposed within the hollow cylindrical body.

### TECHNICAL FIELD

The present invention generally relates to therapeutic devices. More specifically the present invention relates to light or electromagnetic radiation-based physiotherapy devices which may also be used for recreational purposes.

### BACKGROUND ART

Physiotherapy has been known in the art for a quite a while now as a beneficial alternative or an augmenter to conventional medicine. One of the techniques quite commonly deployed in physiotherapy is irradiation therapy, popularly known as light therapy. There are numerous devices in the market for irradiation therapy, but most of such devices are single purpose devices. Such light therapy devices can not be used in conjunction with other physiotherapy devices such as massage guns. Therefore, the user is forced to resort to purchasing multiple devices for distinct modes of physiotherapy, such as light therapy, TENS, PEMF, heating and cooling.

Therefore, there is a need for a device that overcomes the disadvantages and limitations associated with the prior art and provides a more satisfactory solution.

### OBJECTS OF THE INVENTION

Some of the objects of the invention are as follows:

An object of the invention is to provide an irradiation head that can be used in conjunction with a massage gun as an attachment to the massage gun;

Another object of the invention is to provide an irradiation head which can generate sufficient power for irradiation sources available with the irradiation head, such as the LEDs; and

Yet another object of the invention is to provide an irradiation head that is simple in construction and economical for a user.

### SUMMARY OF THE INVENTION

According to a first aspect of the present invention, there is provided an irradiation head adapted to be detachably attached to a massage gun, the irradiation head being configured to reciprocate in forward and backward motion, the irradiation head comprising one or more irradiation sources, and a converter configured to convert the forward and backward motion of the irradiation head into electrical energy, wherein the one or more irradiation sources are configured to be activated by the converted electrical energy, wherein the converter includes a coil based linear alternator, the coil based linear alternator including a coil wrapped around a hollow cylindrical body, and a magnet disposed within the hollow cylindrical body.

In one embodiment of the invention, the converter is connected with the one or more irradiation sources via a bridge rectifier.

In one embodiment of the invention, the irradiation head further comprises an energy storage device adapted to store electrical energy generated by the converter, downstream of the bridge rectifier.

In one embodiment of the invention, the energy storage device is a capacitor.

In one embodiment of the invention, the energy storage device is a battery.

In one embodiment of the invention, the battery is selected from a group consisting of Lithium-ion batteries and Nickel-Metal-Hydride batteries.

In one embodiment of the invention, the battery is configured to be charged wirelessly through a wireless charger.

In one embodiment of the invention, the massage gun includes a user interface adapted to receive signals for modification of characteristics of the reciprocating motion of the irradiation head.

In one embodiment of the invention, the characteristics of the reciprocating motion include speed, stroke length, time interval and momentum transfer.

In one embodiment of the invention, the user interface includes a plurality of push buttons.

In one embodiment of the invention, the user interface includes a touchscreen.

In one embodiment of the invention, the user interface includes a communication interface included in a control module.

In one embodiment of the invention, an external handheld device is configured to communicate with the communication interface through wired or wireless means.

In one embodiment of the invention, the irradiation head further comprises a pressure sensor configured to measure the stiffness in the body of a user.

In one embodiment of the invention, the one or more irradiation sources include a plurality of Light Emitting Diodes (LEDs).

In one embodiment of the invention, the irradiation head is further configured to provide additional modes of therapy using the converted electrical power, wherein the additional modes are selected from a group consisting of Transcutaneous Electrical Nerve Stimulation (TENS) therapy, Pulsed Electro-Magnetic Field (PEMF) therapy, heating and cooling.

According to a second aspect of the present invention, there is provided an irradiation head adapted to be detachably attached to a massage gun, the irradiation head comprising one or more irradiation sources, and an energy storage device configured to power the one or more irradiation sources.

In one embodiment of the invention, the energy storage device is a battery.

In one embodiment of the invention, the battery is selected from a group consisting of Lithium-ion batteries and Nickel-Metal-Hydride batteries.

In one embodiment of the invention, the battery is configured to be charged wirelessly through a wireless charger.

In the context of the specification, the term “processor” refers to one or more of microprocessors, a microcontroller, a general-purpose processor, a Field Programmable Gate Array (FPGA) or an Application Specific Integrated Circuit (ASIC), and the like.

In the context of the specification, the phrase “storage memory” refers to one or more of a volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc., or a non-volatile storage memory such as EPROM, EEPROM or flash memory or the like.

In the context of the specification, the phrase “communication interface” refers to a device or a module enabling direct connectivity via wires and connectors such as USB, HDMI, VGA, or wireless connectivity such as Bluetooth or Wi-Fi or Local Area Network (LAN) or Wide Area Network (WAN) implemented through TCP/IP, IEEE 802.x, GSM, CDMA, LTE or other equivalent protocols.

In the context of the specification, the term “historical” in execution of a command refers to anything pertaining to a time instant(s) that is earlier than a time instant of an initiation of the command.

In the context of the specification, the term, “real-time”, refers to without intentional delay, given the processing limitations of hardware/software/firmware involved and the time required to accurately measure/receive/process/transmit data as practically possible.

In the context of this specification, terms like “light”, “radiation”, “irradiation”, “emission” and “illumination”, etc. refer to electromagnetic radiation in frequency ranges varying from the visible light to Infrared (IR) frequencies and wavelength, wherein the range is inclusive of visible light and IR frequencies and wavelengths. It is to be noted here that IR radiation may be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, “Light Emitting Diodes (LEDs)” are envisaged to be characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow, blue and white colors), Surface Mount LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates a sectional view of an irradiation head, in accordance with an embodiment of the present invention;

FIG. 2 illustrates a circuit diagram representing the irradiation head, in accordance with an embodiment of the present invention;

FIG. 3 illustrates a sectional view of the irradiation head, in accordance with another embodiment of the present invention;

FIG. 4 illustrates a circuit diagram representing the irradiation head of FIG. 3;

FIG. 5 illustrates a user using the irradiation head with a massage gun, for providing light therapy to a part of their body;

FIG. 6A illustrates a massage gun in accordance with an embodiment of the user;

FIG. 6B illustrates the massage gun in accordance with another embodiment of the user;

FIG. 7 illustrates a user utilizing the massage gun with the irradiation head including a pressure sensor mounted onto the irradiation head;

FIG. 8 illustrates the irradiation head in accordance with yet another embodiment of the present invention; and

FIG. 9 illustrates the massage head of FIG. 8 being charged wirelessly using a wireless charger.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

It is envisaged that an irradiation head is provided that may be attached with a massage gun for the provision of light therapy. Conventional massage guns impart reciprocating motion to massage heads which then provide vibratory massage therapy to a user. In the present invention, the reciprocating motion of the massage head is being used to power several irradiation LEDs to impart light therapy in conjunction with vibratory massage therapy. In that regard, the present invention deploys a linear alternator to convert the reciprocating motion of the massage head to electrical energy that may then power the irradiation sources. Referring to the drawings, the invention will now be explained in further detail.

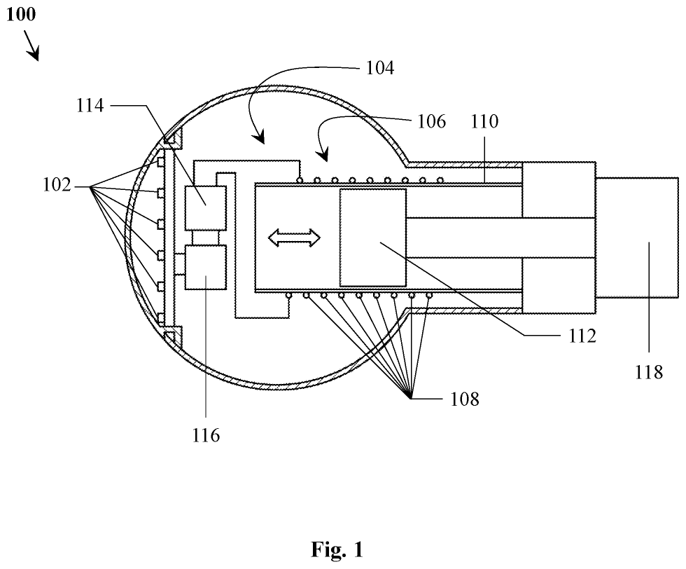

FIG. 1 illustrates a sectional view of an irradiation head 100, in accordance with an embodiment of the present invention. As depicted in FIG. 1, the irradiation head 100 includes one or more irradiation sources 102. In several embodiments of the invention, the one or more irradiation sources 102 include a plurality of Light Emitting Diodes (LEDs). Further, the irradiation head 100 includes a converter 104 configured to convert the forward and backward motion of the irradiation head 100 into electrical energy. In that regard, the converter 104 includes a coil-based linear alternator 106. The coil-based linear alternator 106 includes a coil 108 wrapped around a hollow cylindrical body 110. In several embodiments of the invention, the coil 108 is made up of copper material.

Further, a magnet 112 is freely disposed of within the hollow cylindrical body 108. The magnet 112 may be made up of neodymium material. The magnet 112 in that regard reciprocates freely within the coil 108 leading to generation of alternating electrical current within the coil 108, by Faraday's law of electromagnetic induction. The alternating current produced in the coil 108 is fed to a bridge rectifier 114 and then the direct current thus generated is stored in an energy storage device 116 adapted to store electrical energy generated by the converter 104. In several embodiments of the invention, the energy storage device 116 is a capacitor. In several alternate embodiments, the energy storage device 116 is a battery. In several embodiments of the present invention, the battery is selected from a group consisting of Lithium-ion batteries and Nickel-Metal-Hydride batteries. The one or more irradiation sources 102 are configured to be activated by the converted electrical energy stored in the energy storage device 116.

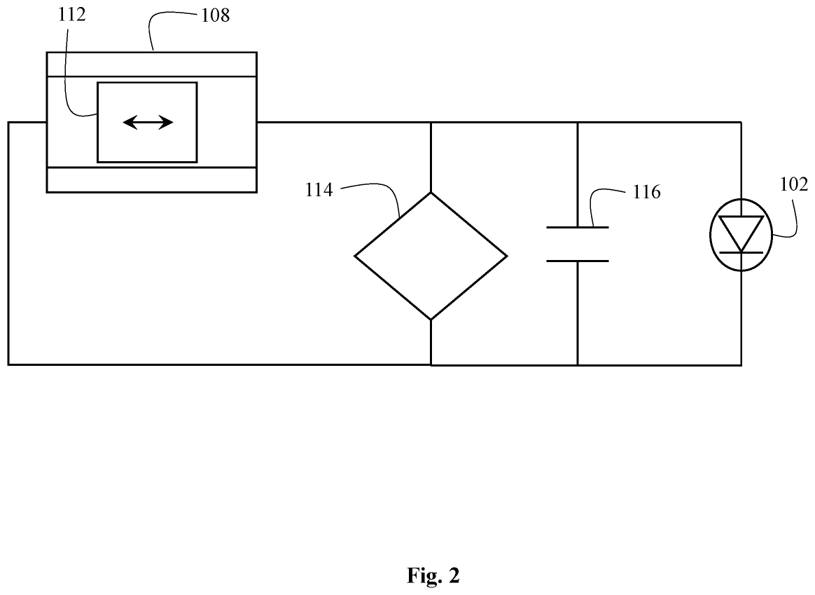

FIG. 2 illustrates a circuit diagram representing the irradiation head 100, in accordance with an embodiment of the present invention. As the magnet 112 reciprocates within the coil 108, due to forward and backward motion of the irradiation head 100, alternating current is produced in the coil 108 by Faraday's law of electromagnetic induction. Since the current is of alternating nature, it cannot be directly fed to the energy storage device 116 and therefore is rectified by the bridge rectifier 114. Once rectified and stored in the energy storage device 116, the DC current may be fed to the one or more irradiation sources 102 to provide the light therapy.

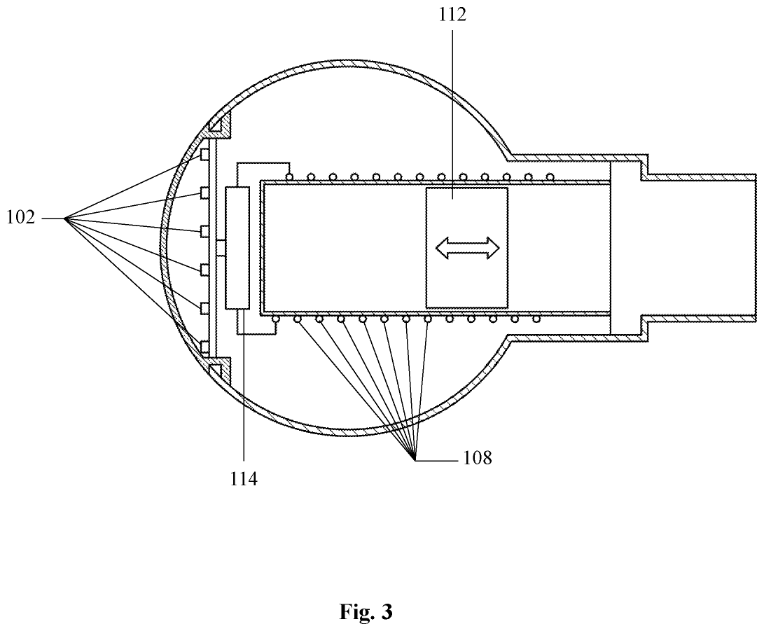

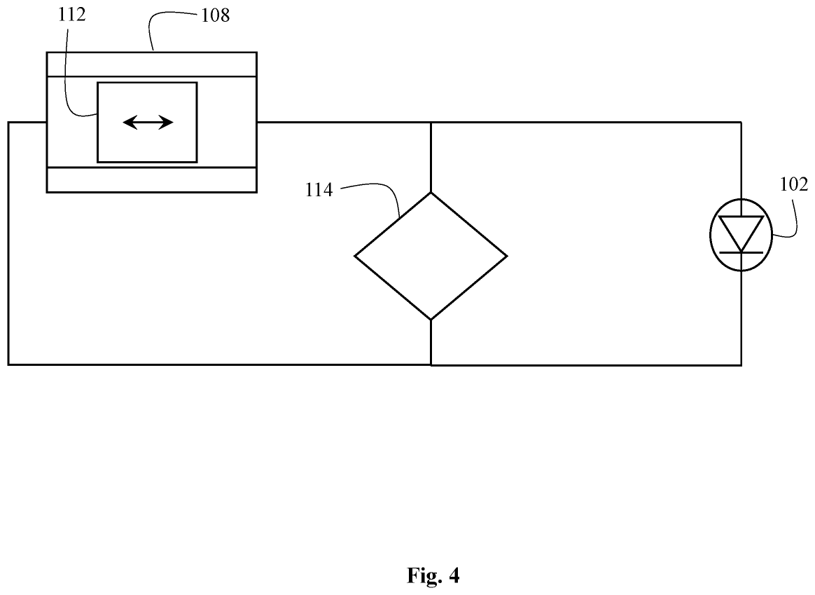

FIG. 3 illustrates the irradiation head in accordance with another embodiment of the present invention. Here the alternating current produced in the coil 108 is fed to a bridge rectifier 114 and then the direct current thus generated directly fed to the one or more irradiation sources 102. FIG. 4 illustrates a circuit diagram representing the irradiation head 100 of FIG. 3. Here the DC current produced by the bridge rectifier 114 is being directly fed to the one or more irradiation sources 102.

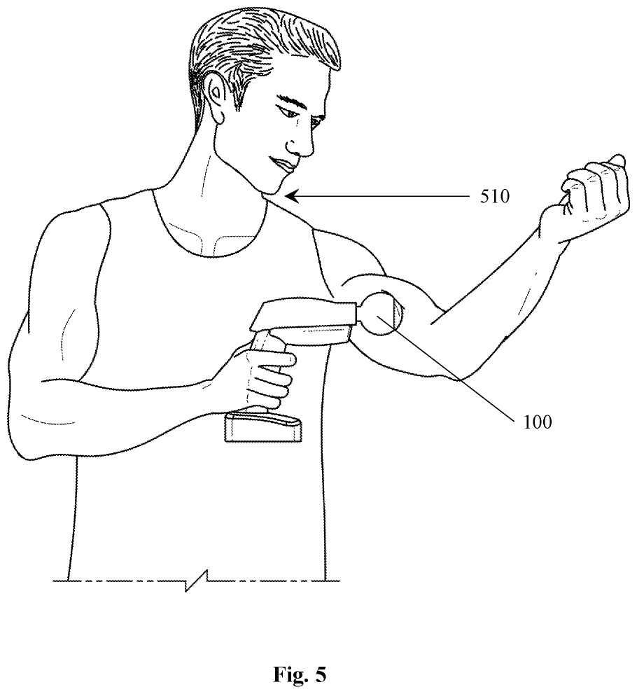

FIG. 5 illustrates a user 510 using the irradiation head 100 with a massage gun, for providing light therapy to a part of their body. It is further to be noted that, in addition to providing light therapy, the irradiation head 100 may also be able to perform other modes of therapies such as Transcutaneous Electrical Nerve Stimulation (TENS) therapy, Pulsed Electro-Magnetic Field (PEMF) therapy, heating, and cooling.

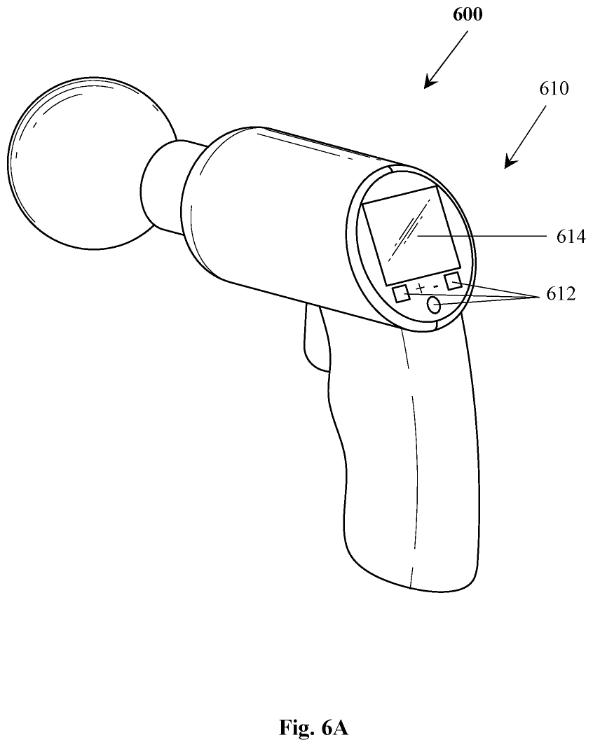

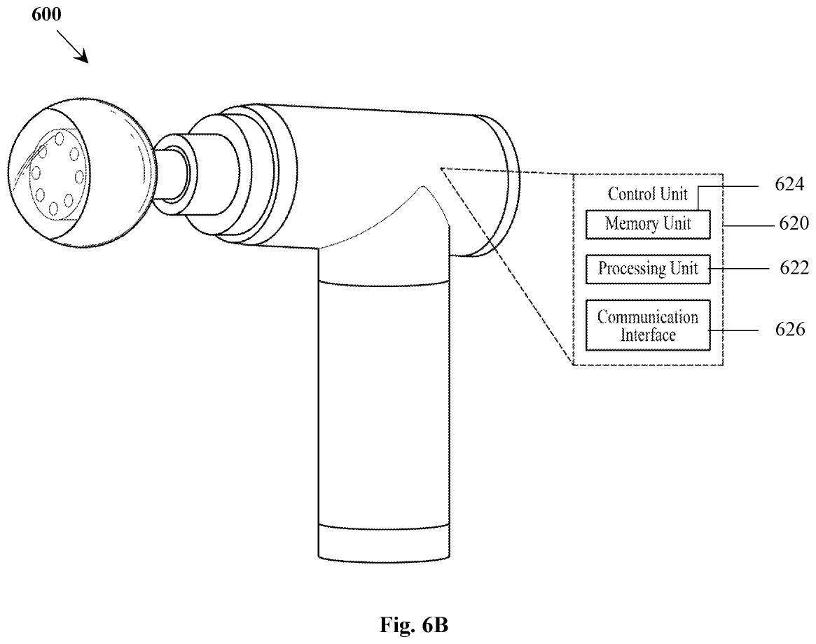

FIG. 6A illustrates a massage gun 600, in accordance with an embodiment of the present invention. The massage gun 600 includes a user interface 610 is adapted to receive input signals for modification of characteristics of the reciprocating motion of the irradiation head 100. The characteristics may include, for example, speed, stroke length, time interval and momentum transfer, etc. In that regard the user interface 610 may include a plurality of push buttons 612. In several embodiments, the user interface 610 may also include a touchscreen 614.

FIG. 6B illustrates a massage gun 600, in accordance with another embodiment of the present invention. The massage gun 600 of FIG. 6B includes a control unit 620 including a processor 622, a memory unit 624 and a communication interface 626. In several embodiments of the invention, the communication interface 626 acts as a user interface by connecting with an external handheld device through wired or wireless means. In that regard, the control unit 620 of the massage gun 600 might receive input signals from the external handheld device through the communication interface 626.

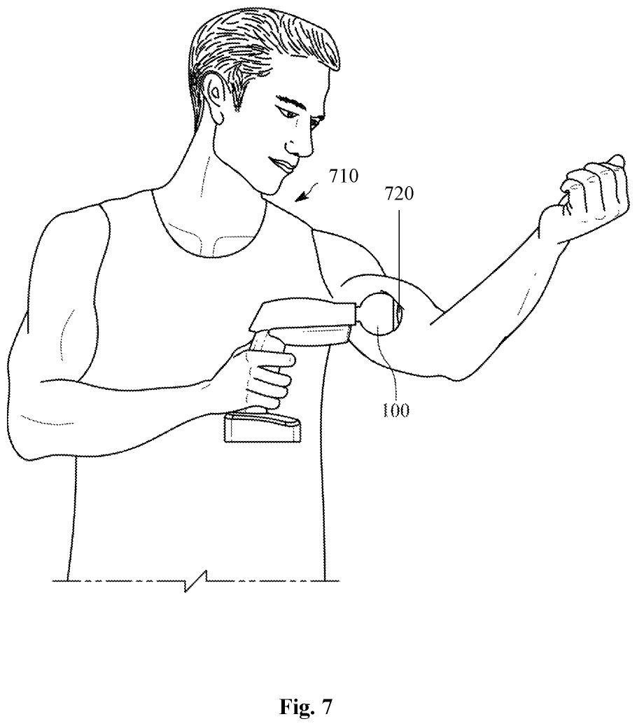

FIG. 7 illustrates a user using the massage gun with the irradiation head 100. There is a pressure sensor 710 located at the front end of the irradiation head 100. The pressure sensor 710 is configured to measure the stiffness in the body of the user before and after the therapy. In that manner, the user will be able to gauge how much effect the irradiation therapy has had on the body of the user. In several embodiments, the stiffness is determined by the control unit 620 of the massage gun 600, in correlation with the signals received from the pressure sensor. Further, the stiffness value is either displayed on the touchscreen 614 or relayed to the external handheld device through the communication interface 626. In addition, the control unit 620 is further configured to measure blood flow in a region of the body of user through diffraction pattern of the Infrared (IR) radiation emitted by the one or more irradiation sources 102. In that manner, the user will be able to assess how much change or difference, in terms of blood flow has the irradiation therapy caused in the region of the body to which the irradiation therapy is being applied.

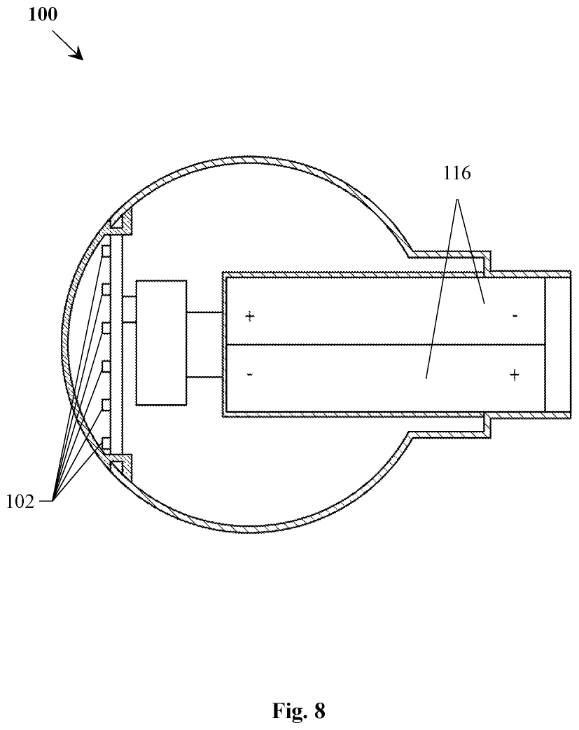

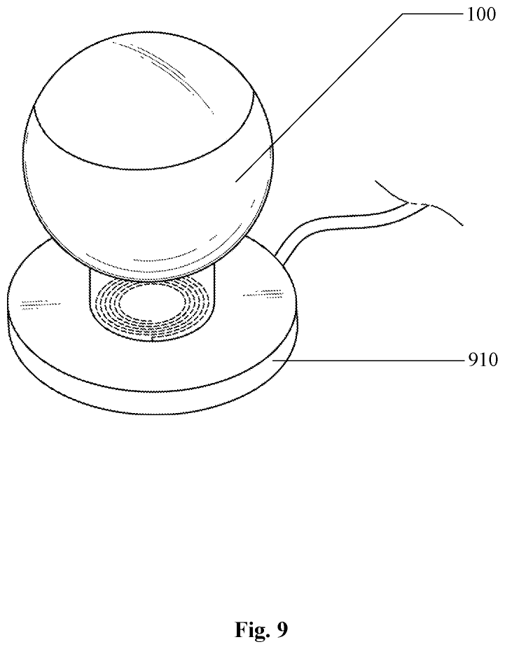

FIG. 8 illustrates the irradiation head in accordance with an alternate embodiment of the present invention. The irradiation head 100 as depicted in FIG. 8 includes the one or more irradiation sources 102 and an energy storage device 116 configured to power the one or more irradiation sources 102. In several embodiments of the present invention, the energy storage device 116 is a battery or a pack of batteries selected from a group consisting of rechargeable Lithium-ion batteries and Nickel-Metal-Hydride batteries. Moreover, the pack of batteries can be charged through wired means or through wireless means as depicted in FIG. 9 using a wireless charger 910.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing the broadest scope consistent with the principles and the novel and inventive features disclosed or suggested herein. Accordingly, the invention is anticipated to hold on to all other such alternatives, modifications, and variations that fall within the scope of the present invention and appended claims.

1. An irradiation head adapted to be detachably attached to a massage gun, the irradiation head being configured to reciprocate in forward and backward motion, the irradiation head comprising:
one or more irradiation sources; and
a converter configured to convert the forward and backward motion of the irradiation head into electrical energy;
wherein the one or more irradiation sources are configured to be activated by the converted electrical energy;
wherein the converter includes a coil based linear alternator, the coil based linear alternator including:
a coil wrapped around a hollow cylindrical body, and a magnet disposed within the hollow cylindrical body.

2. The irradiation head as claimed in claim 1, wherein the converter is connected with the one or more irradiation sources via a bridge rectifier.

3. The irradiation head as claimed in claim 2, further comprising an energy storage device adapted to store electrical energy generated by the converter, downstream of the bridge rectifier.

4. The irradiation head as claimed in claim 2, wherein the energy storage device is a capacitor.

5. The irradiation head as claimed in claim 2, wherein the energy storage device is a battery.

6. The irradiation head as claimed in claim 4, wherein the battery is selected from a group consisting of Lithium-ion batteries and Nickel-Metal-Hydride batteries.

7. The irradiation head as claimed in claim 4, wherein the battery is configured to be charged wirelessly through a wireless charger.

8. The irradiation head as claimed in claim 1, wherein the massage gun includes a user interface adapted to receive signals for modification of characteristics of the reciprocating motion of the irradiation head.

9. The irradiation head as claimed in claim 8, wherein the characteristics of the reciprocating motion include speed, stroke length, time interval and momentum transfer.

10. The irradiation head as claimed in claim 8, wherein the user interface includes a plurality of push buttons.

11. The irradiation head as claimed in claim 8, wherein the user interface includes a touchscreen.

12. The irradiation head as claimed in claim 8, wherein the user interface includes a communication interface included in a control unit.

13. The irradiation head as claimed in claim 12, wherein the control unit is further configured to measure blood flow in a region of the body of user through diffraction pattern of the Infrared (IR) radiation emitted by the one or more irradiation sources.

14. The irradiation head as claimed in claim 12, wherein an external handheld device is configured to communicate with the communication interface through wired or wireless means.

15. The irradiation head as claimed in claim 1, further comprising a pressure sensor configured to measure the stiffness in the body of a user.

16. The irradiation head as claimed in claim 1, wherein the one or more irradiation sources include a plurality of Light Emitting Diodes (LEDs).

17. The irradiation head as claimed in claim 1, further configured to provide additional modes of therapy using the converted electrical power, wherein the additional modes are selected from a group consisting of Transcutaneous Electrical Nerve Stimulation (TENS) therapy, Pulsed Electro-Magnetic Field (PEMF) therapy, heating and cooling.

18. An irradiation head adapted to be detachably attached to a massage gun, the irradiation head comprising:
one or more irradiation sources; and
an energy storage device configured to power the one or more irradiation sources.

19. The irradiation head as claimed in claim 18, wherein the energy storage device is a battery.

20. The irradiation head as claimed in claim 19, wherein the battery is configured to be charged wirelessly through a wireless charger.
